(19) United States
(12) Patent Application Publication
(10) Pub. No.: US 2017/0216178 A1
(43) Pub. Date: Aug. 3, 2017

(54) Cosmetic or Dermatological Composition Containing Retinaldehyde and DNA

(71) Applicant: GALEPHAR PHARMACEUTICAL RESEARCH, INC., JUNCOS, PR (US)

(72) Inventors: Arthur M. DEBOECK, Gurabo, PR (US); Gilbert Mouzin, Castre (FR)

(73) Assignee: GALEPHAR PHARMACEUTICAL RESEARCH, INC., JUNCOS, PR (US)

(21) Appl. No.: 15/418,024

(22) Filed: Jan. 27, 2017

Related U.S. Application Data
(60) Provisional application No. 62/288,800, filed on Jan. 29, 2016.

(51) Int. Cl. A61K 8/60; A61K 8/02; A61K 8/04; A61K 8/33; A61Q 1/02

(52) U.S. Cl. CPC A61K 8/606; A61K 8/33; A61Q 1/02; A61K 8/042; A61K 8/0216; A61K 2800/43; A61K 2800/592; A61K 2800/74

(57) ABSTRACT

A dermatological and/or topical composition, which contains retinaldehyde, and DNA.

### FIELD OF THE INVENTION

The present invention relates to a novel tinted dermatological and/or cosmetic topical composition containing a combination of retinaldehyde with deoxyribonucleic acid or its pharmaceutical acceptable salts for treating acne, more preferably juvenile acne, and skin imperfections in acneic patients.

Preferably, the tinted cream of the present invention is matched to the skin color of the patient, thereby immediately masking the acne patients' skin blemishes.

### BACKGROUND OF THE INVENTION

The causes of juvenile acne are complex and not completely understood. At least four pathophysiological events occur inside the hair follicle affected by acne.
An androgen-dependent stimulation activity of the sebaceous gland.
An abnormal keratinization leading to follicular filling with comedo formation.
A proliferation of the acne bacteria Propiobacterium in the follicule
An inflaation

In addition to these four basic mechanisms, genetic factors, stress and poor eating habits can influence the development and severity of acne.

The retentive lesions (comedones) appear earlier in girls: 75% of them having this type of lesion at the age of 12, whereas only 42% of boys have them. These lesions however, disappear faster. Between the ages of 16 and 17 years only 68% of girls and 90% of boys have blackheads. Boys of all ages suffer more severe and numerous lesions than girls.

Boys are more affected than girls by superficial inflammatory lesions. The age of peak incidence for boys is 17 years of age and 14 years of age for girls. (Daniel F Dreno B, F. Poli Descriptive epidemiology of acne in the school population in France Ann Dermatol Venereol 2000; 127 p 273-278).

Anti-acne topical therapy is the first line treatment.

Local antibacterials currently available are clindamycin, erythromycin, tetracycline and benzoyl peroxide, azelaic acid may also be included in this group because it showed an intra-follicular antibacterial activity against P. acnes. The adverse effects of topical antibiotics include erythema, scaling, dryness and skin burns (Collnick H., Cunliffe W J, Berson D. and all Management of acne: a report from a Global Alliance to Improve Outcomes in Acne J Am Acad Dermatol. 2003, 49 (suppl. 1) 1-37. It has been shown that benzoyl peroxide a free radical initiator, may increase the potential for the development of skin cancer in mice.

The main active topical retinoids, alone or in combination, such as Tretinoine, the isotretinoin, adapalene or Tazarotene effectively reduce the number of comedones and inflammatory lesions, but unfortunately they all cause skin irritation.

Topical retinaldehyde presents a significantly higher skin tolerance to retinoids (Guerrero D, Ane M P: Tolerance and interest of retinaldehyde topical irritant dematitis of the face: Bull Esth Dermatol Cosmetol 1996; 4, 83-87.)

Topical retinaldehyde can be part of the treatment of acne due to its comedolytic action which is identical to that of retinoic acid (tretinoin) Fort-Lacoste L, Verscheure Y, Tisne-Versailles J, Navarro R: Comedolytic effect of topical retinaldehyde in the rhino mouse model. Dermatology 1999; 199 (suppl 1) p 33-35.

French patent FR2681784 of Jan. 10, 1991 relates to the use of retinaldehyde and its derivatives for the treatment of acne.

Therefore, there is still a growing need for compositions that avoid the side effects of current treatments: The present invention provides for a more effective treatment containing retinaldehyde (retinal).

### SUMMARY OF THE INVENTION

The present invention provides a skin tone tinted topical composition containing retinaldehyde in combination with deoxyribonucleic acide (DNA HP) useful for the treatment of acne-prone skin and moderate adolescent acne.

### DETAILED DESCRIPTION OF THE PREFERRED EMBODIMENTS

Preferably, the retinaldehyde according to the invention is in all-trans form or as a trans/cis mixture.

The deoxyribonucleic acid used is highly polymerized (HP) especially the DNA (HP DNA) in the form of its sodium salt. The main biological characteristics of the HP DNA are the following:
Moisturizing action: the HP DNA is an excellent moisturizer, at the cellular level, when in contact with humid air, HP DNA attracts a volume of aqueous solution 10,000 times its own weight.
Antioxidant action: by trapping hydrosyl radicals, HP DNA has the advantage of not generating after capture free radicals, a derivative that may alter proximities components (collagen and elastin).

Its antioxident properties are useful in avoiding the oxidation of linoleic acid present in the sebum, but decreased in patients suffering from acne, this rich sebum squalene peroxide and palmitic acid favors inflammation.

Healing Action: it has been demonstrated in mice and rabbits, that after inducing experimental wounds, the presence of HP DNA accelerates healing.

According to one embodiment, the retinaldehyde used is the all trans retinal.

According to one embodiment, the retinaldehyde concentration is comprised between 0.05 and 5% by weight of the composition, preferably between 0.05 and 0.1%, more preferably between 0.1 and 0.2%, most preferably 0.2 and 0.5% by weight or between 0.5% and 1% or between 1% and 2% or between 2 and 5%.

Preferably, the retinaldehyde contains 95% of trans-isomer and 5% iso mother cis.

According to one embodiment, the highly polymerized DNA is a highly polymerized DNA whose molecular weight is comprised between 1,000 and 5,000 KDa, preferably between 1,500 and 4,000 KDa, preferably between 1,750 and 3,000KDa and more preferably around 2,000 KDa.

According to the preferred embodiment, the highly polymerized DNA is preferably included in the composition of the invention between 1 and 5% by weight of the composition, preferably between 0.5 and 3%, more preferably between 0.5 and 2% and most preferably between 0.1 and 1% by weight.

<<DNA>> means deoxyribonucleic acid and its salts and more particularly its alkaline metal salts. Preferably, the DNA used in the composition of the invention is of marine origin, more preferably DNA from species of fish of the salmonidae family (whose common name is salmon). According to one embodiment, the DNA used in this invention is in solid form, preferably in fiber form.

By “Highly polymerized DNA” it is understood a DNA having a molecular weight between 1,000 and 5,000 kDa, preferably between 1,500 and 4,000 kDa, preferably between 1,750 and 3,000 kDa, and most preferably 2,000 kDa. The highly polymerized DNA is obtained according to methods known to those skilled in the art methods (Verge R, David M, STP Pharma Practices, 3 (2), 99-107, 1993). Extraction of highly polymerized DNA by non-denaturing techniques allows to ensure a perfect protection of the molecular structure of DNA (in particular preserves DNA in its double-stranded form), and thus preserves its physiological activity.

According to a preferred embodiment, the highly polymerized DNA used in the composition of the invention presents as alkaline metal salt, preferably the sodium salt.

Preferably, the DNA of the highly polymerized composition of the invention is obtained by using non-denaturing process.

Preferably, the DNA highly polymerized composition of the invention is highly polymerized DNA produced by the company Javenech which is commercially available under the name HPDR® or INTEGRAL DNA®. DNA highly polymerized product by the company Javenech is in the form of sodium salt, is of marine origin (salmon milt) and has a molecular weight of about 2000 kDa.

The dermo-cosmetic composition of the invention contains pigments to provide color, which is intended to quickly hide skin imperfections in the acne patient, especially in adolescents.

In a preferred embodiment, the dyes are shades of beige or black; in particular a light beige to beige dark pigment, such as C.I. 17200, pigment red 33 or 11427 or a black pigment, such as CI 77499. Dye pigment doses are adjusted for the desired color, for example 1 mg to 10 mg per 100 g of composition; for a light beige dose is 1.5 mg.

The form of the cosmetic or pharmaceutical composition of the invention is not particularly limited, and the cosmetic or pharmaceutical composition of the invention may be in any form such as a liquid, a paste, a gel, a solid or a powder. The cosmetic or pharmaceutical composition of the invention is preferably formulated in a cosmetic composition for the skin, and examples thereof include skin lotions, lotions, creams, emulsions, foundations.

When the composition is in aqueous form, in particular as a dispersion, emulsion or aqueous solution, it may comprise an aqueous phase, which may comprise water, a floral water and/or mineral water. The composition is preferably an aqueous composition and then comprises water at a concentration comprised preferably between 5 and 98% by weight, especially 20 to 95% by weight, preferably 50 to 95% by weight, based on the total weight of the composition.

As is known, the composition according to the invention may comprise the usual adjuvants in the field, such as hydrophilic or lipophilic gelling agents, hydrophilic or lipophilic additives, especially cosmetic active or hydrophilic or lipophilic pharmaceuticals, preservatives, thickeners, vitamins, pH adjusters, antioxidants, solvents, fragrances, fillers, pigments, pearlescent agents, UV screening agents, odor absorbers and coloring matter. These adjuvants, depending on their nature, may be introduced into the fatty phase, or into the aqueous phase and/or in the lipid spherules.

The nature and amount of these adjuvants may be selected by the skilled person, based on his general knowledge, so as to obtain the desired form of presentation for the composition. In any event, the skilled person may select all the optional additional compounds and/or their amounts so that the advantageous properties of the composition according to the invention are not, or not substantially, impaired by the envisaged addition.

The compositions according to the invention may be useful to treat infections such as acne, rosacea, seborrheic dermatitis, atopic dermatitis, psoriasis, disorders of hyperpigmentation, pruritus of different aetiologies and/or atopic dermatitis. The compositions according to the invention can also be used for treating hyperpigmented areas, age spots and/or wrinkles.

The present invention also relates to a non-therapeutic method of cosmetic treatment of the skin comprising applying to the skin a composition comprising an effective amount of at least one retinaldehyde and/or one of such isomers and at least one extract DNA containing HP and more particularly as defined above, in leaving this composition in contact and optionally in rinsing.

The examples which follow are in no way intended to limit the scope of this invention, but are provided to illustrate how to use the compounds of this invention. Many other embodiments will be readily apparent to those skilled in the art.

### EXAMPLES

### Formulation Examples

The topical compositions used in the treatment of acne-prone skin and moderate acne are illustrated by the following examples:

### Example 1

### Gel—Cream

| | | |
|---|---|---|
| Retinaldehyde Trans/cis | 0.1 | g |
| DNA HP sodium salt | 0.5 | g |
| Macropol 600 P | 5 | g |
| Paraffin liquid | 5 | g |
| Alcohol 95° | 3 | g |
| Safflower oil | 2 | g |
| Carbopol 940 | 1 | g |
| Tocopheryl acetate | 0.5 | g |
| Sorbic acid | 0.05 | g |
| Butyl hydroxytoluene | 0.02 | g |
| Triethanolamine | 0.015 | g |
| Preservatives: potassium sorbate, | | |
| sodium benzoate Chlorphenesin | | |
| beige coloring | | |
| Purified water QSP | 100 | g |

### Example 2

### Gel

| | | |
|---|---|---|
| Retinaldehyde | 0.05 | g |
| DNA HP | 0.3 | g |
| Glycerin | 2-3 | g |
| Methyl Gluceth-20 | 2-3 | g |
| Dimethicone | 0.8-1 | g |
| Aminomethyl propanol | 0.4-0.6 | g |
| Acrylates/C10-30alkyl acrylate crosspolymer | 0.1-0.3 | g |
| Bis-hydroxyethoxypropyl Dimethicone Beeswax | 0.1-0.3 | g |
| Parahydroxy benzoate | 0.01-0.02 | g |
| Colorant beige | | |
| Purified water, QSP | 100 | g |

### Example 3

### Cream

| | | |
|---|---|---|
| Retinaldehyde | 0.1 | g |
| DNA HP | 1 | g |
| Triglycerides capric/caprylic | 12-16 | g |
| Glycerol monostearate PEG 100 | 7-9 | g |
| Liquid Paraffin | 4-6 | g |
| Squalane | 2-4 | g |
| Propylene glycol | 2-4 | g |
| E wax powder | 1-2 | g |
| Hydrogenated ethoxylated castor oil | 1-2 | g |
| Triethanolamine | 0.5-0.7 | g |
| Carbopol 934 | 0.3-0.5 | g |
| Ethyl parahydroxybenzoate | 0.3-0.5 | g |
| Methyl parahydroxybenzoate | 0.1-0.3 | g |
| Disodium edetate | 0.03-0.05 | g |
| Butylhydroxytoluene | 0.01-0.03 | g |
| Beige colorant | | |
| Purified water, QSP | 100 | g |

### Example 4

### Cream

| | | |
|---|---|---|
| Retinaldehyde | 0.1 | g |
| DNA HP | 0.5 | g |
| Safflower oil | 8-12 | g |
| Neobee M5 | 8-12 | g |
| Glycerol Monostearate | 7-9 | g |
| Liquid Paraffin | 4-6 | g |
| Propylene glycol | 2-4 | g |
| Acetulan | 2-4 | g |
| E wax powder | 1-3 | g |
| Solulan PB10 | 1-3 | g |
| Ethoxylated hydrogenated castor oil | 1-2 | g |
| Methyl Parahydroxybenzoate | 0.3-0.5 | g |
| Triethanolamine | 0.2-0.4 | g |
| Carbopol 934 | 0.2-0.3 | g |
| Ethylene Parahydroxybenzoate | 0.2-0.3 | g |
| Butyl hydroxytoluène | 0.01-0.03 | g |
| Beige colorant | | |
| Purified water, QSP | 100 | g |

### Example 5

### Cream

| | | |
|---|---|---|
| Retinaldehyde | 0.1 | g |
| DNA HP | 0.5 | g |
| Triglycerides/capric/caprylic 30-70 | 7-10 | g |
| Glycerol Monostearate PEG 100 | 4-6 | g |
| Liquid Paraffin | 2-5 | g |
| Propylene glycol | 2-5 | g |
| Squalane | 1-3 | g |
| Sepigel 305 | 0.5-1.5 | g |
| Triethanolamine | 0.3-0.5 | g |
| Methyl Parahydroxybenzoate | 0.3-0.5 | g |
| Ethyl Parahydroxybenzoate | 0.2-0.3 | g |
| Disodium ethetate | 0.04-0.05 | g |
| Butylhydroxytoluene | 0.01-0.03 | g |
| Beige Colorant | | |
| Purified water, QSP | 100 | g |

It may be noted that the anti-acne retinaldehyde of association with DNA HP effect may be potentiated by the use of a tinted cream color of the patient's skin. Indeed it appeared that the Caucasian type of patients who used the formulation according to Example 1 highly appreciated the immediate masking skin imperfections thanks to the beige color of the formulation which may have a positive effect on the psychological stress factor exacerbating acne.

What is claimed is:

1. A dermatological composition and/or topical composition comprising:
(a) retinaldehyde, and
(b) deoxyribonucleic acid.

2. The composition of claim 1, which further comprises one or more pigments allowing immediate masking of skin imperfections.

3. The composition of claim 1, wherein said retinaldehyde comprises all-trans retinaldehyde or a mixture of trans/cis isomers or both.

4. The composition of claim 1, comprising from 0.05% to 0.1% by weight based on the total weight of the composition.

5. The composition of claim 1, wherein the deoxyribonucleic acid is highly polymerized (HP DNA).

6. The composition of claim 1, wherein the HP DNA is in a form of a sodium salt.

7. The composition of claim 5, comprising from 0.1 to 1% and by weight of HP DNA.

8. The composition of claim 2, wherein the one or more pigments are selected to match skin color of a human patient.

9. The composition of claim 8, wherein the one or more pigments are selected from the group consisting of light beige to dark beige pigment.

10. The composition of claim 9, wherein the one or more pigments are selected from the group consisting of C.I. 17200, pigment red 33, 11427 and C.I. 77499.

11. The composition of claim 5, wherein said HP DNA has a molecular weight of between about 1,000 to 5,000 KDa.

12. The composition of claim 11, wherein said HP DNA has a molecular weight of between about 1,750 to 3,000 KDa.

13. The composition of claim 1, wherein said DNA is of marine origin.

14. The composition of claim 13, wherein said DNA is from a species of fish from Salmonidae family.

15. The composition of claim 14, wherein the DNA is in a form of a sodium salt with a molecular weight of about 2,000 KDa.

16. The composition of claim 1, which is in a form of an aqueous solution, cream, lotion, emulsion or powder.

17. The composition of claim 1, which further comprises an adjuvant or excipient.

18. The composition of claim 17, wherein the adjuvant or excipient comprises hydrophilic or lipophilic gelling agents, hydrophilic or lipophilic additives, preservatives, thickeners, vitamins, pH adjusters, antioxidants, solvents, fragrances, fillers, pigments, pearlescent agents, UV screening agents, odor absorbers and/or coloring matter.

19. The composition of claim 1, which is in a form of a cosmetic.

20. A method of treating juvenile or moderate acne in a human, which comprises administering an effective amount of the composition of claim 1, to a human in need thereof.

21. A method of treating acne-prone skin and skin imperfections, which comprises administering an effective amount of the composition of claim 1, to a human in need thereof.
